(19) United States
(12) Patent Application Publication
(10) Pub. No.: US 2026/0027372 A1
(43) Pub. Date: Jan. 29, 2026

(54) NEGATIVE POISSON'S RATIO MATERIALS FOR ELECTRICAL DEVICES

(71) Applicant: Joon Bu Park, Las Vegas, NV (US)

(72) Inventor: Joon Bu Park, Las Vegas, NV (US)

(21) Appl. No.: 18/787,151

(22) Filed: Jul. 29, 2024

(51) Int. Cl. A61N 1/375; A61N 1/362; A61N 1/378; H01M 50/124; H01M 50/136

(52) U.S. Cl. CPC A61N 1/37512; A61N 1/362; A61N 1/378; H01M 50/124; H01M 50/136; H01M 2220/30

(57) ABSTRACT

A battery includes a frame defining an interior space. The frame includes an inner layer including a material having a negative Poisson's ratio (NPR material), and an outer layer disposed on the inner layer such that the inner layer faces the interior space and the outer layer faces an exterior of the frame. The battery includes an electrolyte contained in the interior space of the frame; a positive electrode disposed in the interior space of the frame, the positive electrode including a positive electrode active material; and a negative electrode disposed in the interior space of the frame, the negative electrode including a negative electrode active material.

### BACKGROUND

Materials with a negative Poisson's ratio (“NPR materials”) respond to compression along one direction by undergoing compression in the two perpendicular directions. NPR materials can exhibit various desirable properties, including high shear modulus, effective energy absorption, and high toughness (e.g., high resistance to indentation, high fracture toughness), among others.

### SUMMARY

We describe here batteries, pacemakers, and wires that include materials having a negative Poisson's ratio (“NPR materials”), in some examples in composites with materials having a positive Poisson's ratio (“PPR materials”). The incorporation of NPR materials into these devices advantageously captures the low-density advantages of NPR materials, which can translate into lighter devices, high strength-to-weight ratios, and high surface area. In the context of NPR-PPR composites, PPR materials can provide complementary advantages, such as hardness, water resistance, and biocompatibility.

In a first aspect, a battery includes a frame defining an interior space. The frame includes an inner layer including a material having a negative Poisson's ratio (NPR material), and an outer layer disposed on the inner layer such that the inner layer faces the interior space and the outer layer faces an exterior of the frame. The battery includes an electrolyte contained in the interior space of the frame; a positive electrode disposed in the interior space of the frame, the positive electrode including a positive electrode active material; and a negative electrode disposed in the interior space of the frame, the negative electrode including a negative electrode active material.

Embodiments can include one or any combination of two or more of the following features.

The outer layer of the frame includes a material having a positive Poisson's ratio (PPR material).

The outer layer of the frame includes an NPR material.

The inner layer of the frame includes a composite material including the NPR material and a PPR material. In some cases, the composite material of the inner layer includes a matrix of the PPR material with the NPR material embedded therein. In some cases, the composite material of the inner layer includes a matrix of the NPR material with the PPR material embedded therein. In some cases, the composite material of the inner layer includes a layered composite including alternating layers of the PPR material and the NPR material.

The inner layer of the frame has a negative Poisson's ratio.

The positive electrode active material has a negative Poisson's ratio. In some cases, the negative electrode active material has a negative Poisson ratio.

The positive electrode active material includes a lithium metal oxide or a lithium metal phosphate.

The negative electrode active material includes graphite, graphene, or a nanostructured carbon material.

The battery includes a porous membrane disposed in the interior space between the positive electrode and the negative electrode. In some cases, the porous membrane includes an NPR material.

In a second aspect, combinable with the first aspect, a pacemaker includes a hermetic housing including a material having a negative Poisson's ratio (NPR material); a power pack disposed in an interior of the hermetic housing, wherein the power pack is configured to generate electrical pulses; and an electrode, wherein a first end of the electrode is electrically connected to the power pack and a second end of the electrode extends through the hermetic housing to an exterior of the pacemaker, the second end of the electrode configured to be connected to tissue of a patient.

Embodiments can include one or any combination of two or more of the following features.

The hermetic housing includes a composite material including the NPR material and a PPR material. In some cases, the composite material of the hermetic housing includes a matrix of the PPR material with the NPR material embedded therein. In some cases, the composite material of the hermetic housing includes a matrix of the NPR material with the PPR material embedded therein. In some cases, the composite material of the hermetic housing includes a layered composite including alternating layers of the PPR material and the NPR material. In some cases, an outer surface of the hermetic housing is formed of PPR material.

The electrode includes an NPR material.

The electrode includes a core including the NPR material; and a coating surrounding a length of the core, the coating including a PPR material.

A distal tip of the electrode includes an NPR material.

### BRIEF DESCRIPTION OF DRAWINGS

FIG. 1 is a diagram of a battery.

FIG. 2 is a diagram of a battery.

FIG. 3 is an illustration of materials with negative and positive Poisson's ratios.

FIG. 4 is a diagram of NPR-PPR composite materials.

FIG. 5 is a diagram of PPR materials and NPR-PPR composite materials.

FIG. 6 is a diagram of a pacemaker.

FIG. 7 is a diagram of a pacemaker.

FIGS. 8-9 are diagrams of electrodes.

FIGS. 10A and 10B are a side cross sectional view and a cross sectional view, respectively, of an electrode.

### DETAILED DESCRIPTION

We describe here batteries, pacemakers, and wires that include materials having a negative Poisson's ratio (“NPR materials”), in some examples in composites with materials having a positive Poisson's ratio (“PPR materials”). The incorporation of NPR materials into these devices advantageously captures the low-density advantages of NPR materials, which can translate into lighter devices, high strength-to-weight ratios, and high surface area. In the context of NPR-PPR composites, PPR materials can provide complementary advantages, such as hardness, water resistance, and biocompatibility.

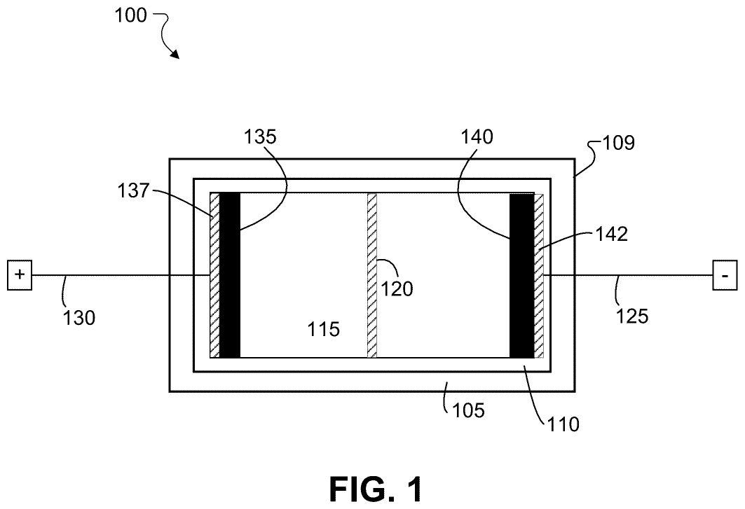

Referring to FIG. 1, a battery 100, such as a rechargeable lithium ion battery, includes one or more components that include an NPR material. The battery 100 has a frame 109 that defines an interior space 115 that contains an electrolyte, positive and negative electrodes 130, 135, and a porous membrane 120.

The frame 109 has an inner layer 110 facing the interior space 115 and an outer layer 105 facing an exterior of the battery. The outer layer 105 of the frame acts as a casing for the battery 100 that encloses and protects the internal components of the battery 100. In some examples, the outer layer 105 includes metals, such as steel or aluminum. In some examples, the outer layer 105 includes plastic or rubber. In some examples, the outer layer 105 includes NPR material. The inclusion of NPR material in the outer layer 105 allows the frame to be lighter in weight than conventional frames composed of PPR materials, while providing improved stress response characteristics that can enhance the durability, performance, and/or longevity of the battery 100.

The inner layer 110 of the frame provides structural support and environmental isolation to the components in the interior 115 of the battery and is composed of a material that is non-reactive with the electrolyte and non-electroactive. The inner layer 110 includes an NPR material. Inclusion of NPR material in the inner layer 110 of the frame renders the frame less dense (and thus lighter in weight) as compared to frames formed entirely of PPR materials, which retaining the structural (e.g., dimensional) stability provided by the frame. In some examples, the inner layer 110 also includes metals, such as zinc, lithium, manganese, cobalt, aluminum, or copper. In some examples, the inner layer 110 includes PPR materials, creating an inner layer 110 with synergistic benefits of combined NPR-PPR materials. Similar to the outer layer 105, some NPR materials are porous and lack hardness. However, an inner layer 110 with PPR material can be non-porous and have a high hardness so as to wholly or partially mitigate potential drawbacks of NPR material inclusion. In operation of the battery 100, the inner layer 110 of the battery 100 protects the internal components of the battery 100.

In some examples, either or both of the inner layer 110 or the outer layer 105 of the frame is formed of a composite material including both NPR material and PPR material (an “NPR-PPR composite). Such composites can be matrix composites, laminar composites, fiber composites, or other appropriately structured composites, e.g., as discussed further below. The use of NPR-PPR composite materials in the frame provides the frame with advantages stemming from the use of both NPR and PPR materials. For instance, while NPR materials are lightweight, they can be porous and lack hardness, while PPR materials are often harder and less porous. The inclusion of both NPR and PPR materials in the frame allows the density advantages of NPR materials to be achieved while also presenting a non-porous (e.g., water resistant), hard structure via the PPR material of the composite.

In a specific example, the inner layer 110 is a layered NPR-PPR composite with PPR material forming the innermost layer that faces the interior space 115 of the battery. In this arrangement, the low density of the NPR material renders the frame lightweight, while the PPR material that faces the interior space 115 of the battery exposes a non-porous and chemically robust surface to the electrolyte. Similarly, in another specific example, the outer layer 105 is a layered NPR-PPR composite with PPR material forming the outermost layer that faces the exterior of the battery. In this arrangement, the low density of the NPR material renders the frame lightweight, while the PPR material that faces the exterior of the battery exposes a hard, non-porous to the exterior environment of the battery. Moreover, in this configuration, when the PPR material is a biocompatible material having low thrombogenicity and low toxicity, the composite housing obtains the strength and weight advantages from the NPR material while presenting a biocompatible surface to the exterior (e.g., for exposure to the patient) that is unlikely to promote an adverse immune response.

A negative electrode 140 composed of negative electrode active material is disposed in the interior space 115 of the battery 100. In some examples, the negative electrode active material is a carbon-based material such as graphite, graphene, nanostructured carbon (e.g., carbon nanotubes), or another suitable carbon-based material. Other suitable negative electrode active materials can also be used, such as silicon-based materials, titanium dioxide, or other negative electrode active materials. The negative electrode 140 also includes a binder, such as a polymeric binder.

The negative electrode 140 is disposed on a current collector 142, such as a conductive foil, e.g., a metal foil such as copper foil; a conductive mesh; a conductive foam; or a conductive coating. The current collector 142 is electrically connected to the exterior of the battery 100 via an electrode lead 125 that extends through the frame 109.

A positive electrode 135 composed of positive electrode active material is also disposed in the interior space 115 of the battery 100. In some examples, the positive electrode active material is a lithium metal oxide or a lithium metal phosphate, such as lithium cobalt oxide (LiCoO2), lithium manganese oxide (LiMn2O4), lithium iron phosphate (LiFePO4), lithium nickel manganese cobalt oxide (LiNiMnCoO2), or another suitable positive electrode active material. The positive electrode 135 also includes a binder, such as a polymeric binder.

The positive electrode 135 is disposed on a current collector 137, such as a conductive foil, e.g., a metal foil such as copper foil; a conductive mesh; a conductive foam; or a conductive coating. The current collector 137 is electrically connected to the exterior of the battery 100 via an electrode lead 130 that extends through the frame 109.

In some examples, the positive electrode 135 and/or the negative electrode 140 include an NPR material. For instance, the lithium metal oxide or phosphate of the positive electrode 135 can be an NPR material and/or the carbon-based material of the negative electrode 140 can be an NPR material. The porosity of the NPR material increases the surface area of the electrodes as compared to conventional PPR electrodes without sacrificing mechanical strength/integrity, thus promoting efficient reactions and reducing weight of the battery.

A porous membrane 120 that acts as a separator is disposed in the interior space 115 between the positive electrode 135 and the negative electrode 140. The separator 120 is a mechanically robust layer that prevents physical and electrical contact between the positive and negative electrodes 135, 140. The separator 120 is porous to enable ion transport therethrough. In some examples, the separator 120 is formed of a thermally conductive material to facilitate thermal management in the battery 100. Examples of materials for the separator 120 include polymer sheets, e.g., polyolefin sheets such as polyethylene or polypropylene, other types of polymer sheets such as polyethylene terephthalate or polyvinylidene fluoride, or other suitable materials such as cellulose based materials.

In some examples, the porous membrane 120 includes an NPR material, which enhances the porosity of the membrane as compared to a PPR membrane while enabling mechanical strength and structural integrity of the membrane to be retained.

In the example illustrated in FIG. 1, the positive electrode 135 and the negative electrode 140 are disposed on opposite sides of the battery, e.g., separated by the width of the interior space, and the separator extends across the entire height and depth of the interior space. Other arrangements are also possible. For instance, the positive electrode 135 and the negative electrode 140 can be interdigitated. In some examples, the battery 100 can be a jelly roll style battery in which the positive and negative electrodes 135, 140 and the separator 120 are rolled into a cylindrical structure.

The electrolyte contained in the interior space 115 can be a solid electrolyte or a liquid electrolyte suitable for conducting ions (e.g., lithium ions). Examples of suitable electrolytes include polymer electrolytes such as polyvinyl alcohol, polyacrylonitrile, polyethylene glycol, or polyvinyl butyral; ceramic based electrolytes, sulfide based electrolytes, or other suitable lithium ion battery electrolytes.

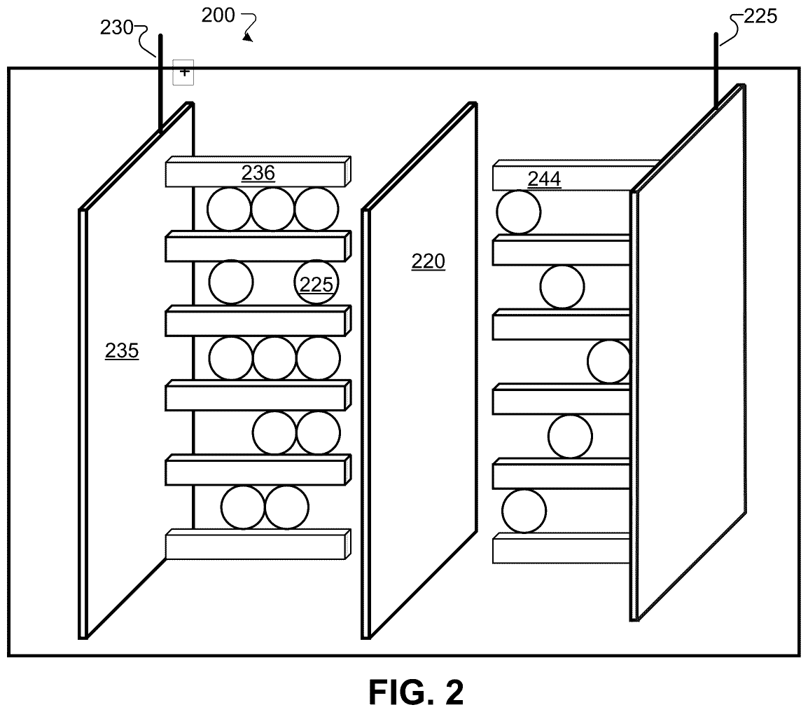

NPR materials can be incorporated into lithium ion batteries having structures other than those illustrated in FIG. 1. Referring to FIG. 2, a fast charge lithium ion battery 200 includes a housing 209 that can include NPR materials, e.g., NPR-PPR composite materials, e.g., as described above for the battery 100 of FIG. 1.

The fast charge battery 200 positive electrode fingers 236 and negative electrode fingers 244 connected to respective positive and negative electrode panels 235, 240. The electrode fingers and electrode panels include the respective positive or negative electrode active material disposed on a current collector (not illustrated), such as a conductive foil or other suitable substrate. The positioning of the positive and negative electrode fingers 236, 244 means that lithium ions 225 do not need to travel a long distance during charging/discharging, which allows for rapid cycling of the battery. A porous membrane separator 220 is disposed between ends of the positive electrode fingers 236 and ends of the negative electrode fingers 244.

In the fast charge battery 200, the positive and/or negative electrode active material of the electrode fingers 236, 244 and electrode panels 235, 240 and/or the separator 220 can include NPR materials, e.g., as described above for the battery 100 of FIG. 1.

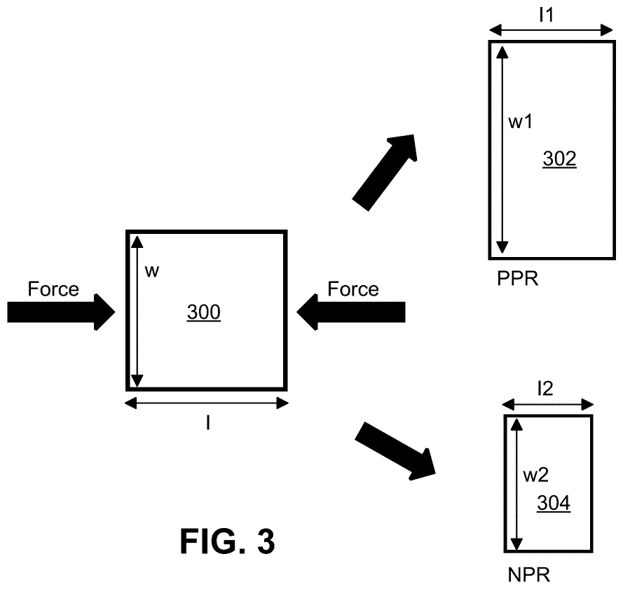

Materials with negative and positive Poisson's ratios are illustrated in FIG. 3, which depicts a hypothetical two-dimensional block of material 300 with length 1 and width w.

If the hypothetical block of material 300 is a PPR material, when the block of material 300 is compressed along its width w, the material deforms into the shape shown as block 302. The width w1 of block 302 is less than the width w of block 300, and the length 11 of block 302 is greater than the length 1 of block 300: the material compresses along its width and expands along its length.

By contrast, if the hypothetical block of material 300 is an NPR material, when the block of material 300 is compressed along its width w, the material deforms into the shape shown as block 304. Both the width w2 and the length 12 of block 304 are less than the width w and length 1, respectively, of block 300: the material compresses along both its width and its length.

NPR materials can exhibit various desirable properties, including high shear modulus, effective energy absorption, and high toughness (e.g., high resistance to indentation, high fracture toughness), among others. The properties of NPR materials are such that an item that includes an NPR material undergoes a different (e.g., smaller) change in dimension when absorbing energy than a comparable item formed of only PPR material.

NPR materials for integration into batteries, e.g., for use in the battery frame, electrodes, or membranes, can be foams, such as polymeric foams, ceramic foams, metal foams, or combinations thereof. A foam is a multi-phase composite material in which one phase is gaseous and the one or more other phases are solid (e.g., polymeric, ceramic, or metal). Foams can be closed-cell foams, in which each gaseous cell is sealed by solid material; open-cell foams, in which the each cell communicates with the outside atmosphere; or mixed, in which some cells are closed and some cells are open. In some examples, NPR materials can be foams composed of micro- or nano-tubules. When the battery includes NPR foams, the composition can be that of a conventional layer, with the structure of the foam being such that the material has a negative Poisson's ratio. Using an NPR foam in the battery provides enhanced energy absorption and greater impact resistance, creating more durable batteries.

An example of an NPR foam structure is a re-entrant structure, which is a foam in which the walls of the cells are concave, e.g., protruding inwards toward the interior of the cells. In a re-entrant foam, compression applied to opposing walls of a cell will cause the four other, inwardly directed walls of the cell to buckle inward further, causing the material in cross-section to compress, such that a compression occurs in all directions. Similarly, tension applied to opposing walls of a cell will cause the four inwardly directed walls of the cell to unfold, causing the material in cross-section to expand, such that expansion occurs in all directions. NPR foams can have a Poisson's ratio of between −0.5 and 0, e.g., −0.5, −0.4, −0.3, −0.2, or −0.1. NPR foams can have an isotropic Poisson's ratio (e.g., Poisson's ratio is the same in all directions) or an anisotropic Poisson's ratio (e.g., Poisson's ratio when the foam is strained in one direction differs from Poisson's ratio when the foam is strained in a different direction).

An NPR foam can be polydisperse (e.g., the cells of the foam are not all of the same size) and disordered (e.g., the cells of the foam are randomly arranged, as opposed to being arranged in a regular lattice). An NPR foam can be a cellular structure having a characteristic dimension (e.g., the size of a representative cell, such as the width of the cell from one wall to the opposing wall) ranging from 0.1 μm to about 3 mm, e.g., about 0.1 μm, about 0.5 μm, about 1 μm, about 10 μm, about 50 μm, about 100 μm, about 500 μm, about 1 mm, about 2 mm, or about 3 mm.

In some examples, NPR foams are produced by transformation of PPR foams to change the structure of the foam into a structure that exhibits a negative Poisson's ratio. In some examples, NPR foams are produced by transformation of nanostructured or microstructured PPR materials, such as nanospheres, microspheres, nanotubes, microtubes, or other nano- or micro-structured materials, into a foam structure that exhibits a negative Poisson's ratio. The transformation of a PPR foam or a nanostructured or microstructured material into an NPR foam can involve thermal treatment (e.g., heating, cooling, or both), application of pressure, or a combination thereof. In some examples, PPR materials, such as PPR foams or nanostructured or microstructured PPR materials, are transformed into NPR materials by chemical processes, e.g., by using glue. In some examples, NPR materials are fabricated using micromachining or lithographic techniques, e.g., by laser micromachining or lithographic patterning of thin layers of material. In some examples, NPR materials are fabricated by additive manufacturing (e.g., three-dimensional (3D) printing) techniques, such as stereolithography, selective laser sintering, or other appropriate additive manufacturing technique.

In an example, a PPR thermoplastic foam, such as an elastomeric silicone film, can be transformed into an NPR foam by compressing the PPR foam, heating the compressed foam to a temperature above its softening point, and cooling the compressed foam. In an example, a PPR foam composed of a ductile metal can be transformed into an NPR foam by uniaxially compressing the PPR foam until the foam yields, followed by uniaxially compression in other directions.

In some examples, NPR foams are produced by transformation of PPR foams to change the structure of the foam into a structure that exhibits a negative Poisson's ratio. In some examples, NPR foams are produced by transformation of nanostructured or microstructured PPR materials, such as nanospheres, microspheres, nanotubes, microtubes, or other nano- or micro-structured materials, into a foam structure that exhibits a negative Poisson's ratio. The transformation of a PPR foam or a nanostructured or microstructured material into an NPR foam can involve thermal treatment (e.g., heating, cooling, or both), application of pressure, or a combination thereof. In some examples, PPR materials, such as PPR foams or nanostructured or microstructured PPR materials, are transformed into NPR materials by chemical processes, e.g., by using glue. In some examples, NPR materials are fabricated using micromachining or lithographic techniques, e.g., by laser micromachining or lithographic patterning of thin layers of material. In some examples, NPR materials are fabricated by additive manufacturing (e.g., three-dimensional (3D) printing) techniques, such as stereolithography, selective laser sintering, or other appropriate additive manufacturing technique.

In an example, a PPR thermoplastic foam, such as an elastomeric silicone film, can be transformed into an NPR foam by compressing the PPR foam, heating the compressed foam to a temperature above its softening point, and cooling the compressed foam. In an example, a PPR foam composed of a ductile metal can be transformed into an NPR foam by uniaxially compressing the PPR foam until the foam yields, followed by uniaxially compression in other directions.

In some examples, the battery frame, electrodes, and/or membrane are formed of NPR-PPR composite materials. NPR-PPR composite materials are composites that include both regions of NPR material and regions of PPR material. NPR-PPR composite materials can be laminar composites, matrix composites (e.g., metal matrix composites, polymer matrix composites, or ceramic matrix composites), particulate reinforced composites, fiber reinforced composites, or other types of composite materials. In some examples, the NPR material is the matrix phase of the composite and the PPR material is the reinforcement phase, e.g., the particulate phase or fiber phase. In some examples, the PPR material is the matrix phase of the composite and the NPR material is the reinforcement phase.

NPR materials can exhibit various desirable properties, including high shear modulus, effective energy absorption, and high toughness (e.g., high resistance to indentation, high fracture toughness), among others. The properties of NPR materials are such that an item that includes an NPR material undergoes a different (e.g., smaller) change in dimension when absorbing energy than a comparable item formed of only PPR material.

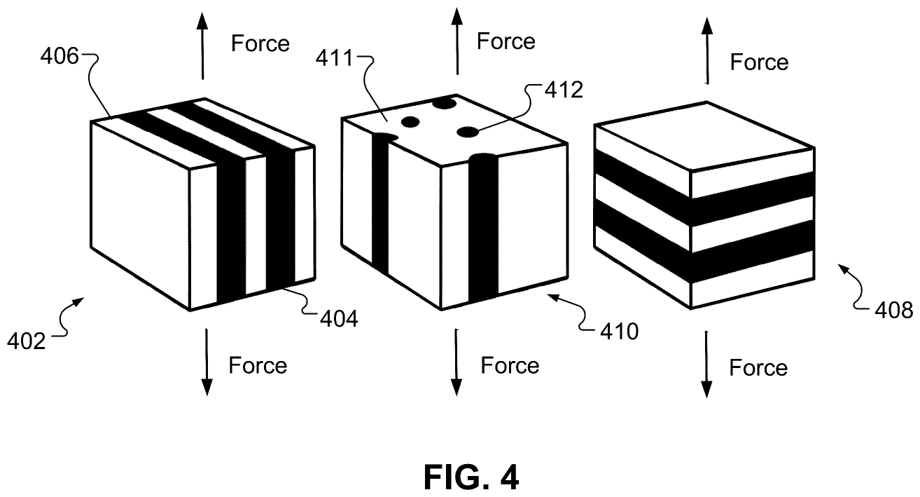

FIG. 4 illustrates examples of NPR-PPR composite materials. An NPR-PPR composite material 402 is a laminar composite including alternating layers 404 of NPR material and layers 406 of PPR material. The layers 404, 408 are arranged in parallel to a force to be exerted on the composite material 402. Although the layers 404, 406 are shown as having equal width, in some examples, a laminar composite can have layers of different widths.

An NPR-PPR composite material 408 is a laminar composite including alternating layers of NPR material and PPR material, with the layers arranged perpendicular to a force to be exerted on the material 408. In some examples, the layers of a laminar composite are arranged at an angle to the expected force that is neither perpendicular nor parallel.

An NPR-PPR composite material 412 is a matrix composite including a matrix phase 411 of NPR material with a reinforcement phase 412 of PPR material. In the material 412, the reinforcement phase 412 includes fibers of the PPR material; in some examples, the reinforcement phase 412 can include particles or other configuration. In some examples, NPR-PPR composite materials can have a matrix phase of a PPR material with a reinforcement phase of an NPR material.

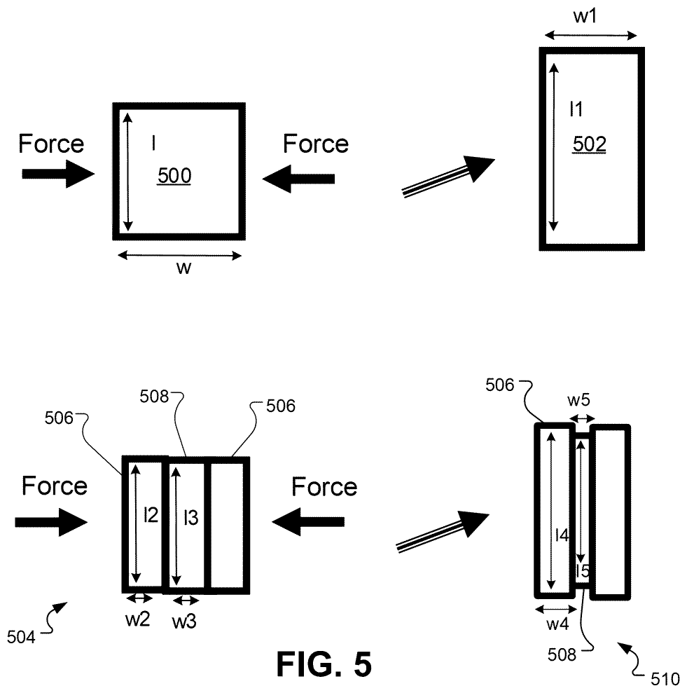

FIG. 5 illustrates the mechanical behavior of PPR and NPR/PPR composite materials. A hypothetical block 500 of PPR material, when compressed along its width w, deforms into a shape 502. The width w1 of the compressed block 502 is less than the width w of the uncompressed block 500, and the length 11 of the compressed block 502 is greater than the length 1 of the uncompressed block: the material compresses along the axis to which the compressive force is applied and expands along a perpendicular axis.

A block 504 of NPR/PPR composite material includes a region 508 of NPR material sandwiched between two regions 506 of PPR material. When the block 504 of composite material is compressed along its width, the material deforms into a shape 510. The PPR regions 506 compress along the axis of compression and expand along a perpendicular axis, e.g., as described above for the block 500 of PPR material, such that, e.g., the width w2 of a region 506 of uncompressed PPR material compresses to a smaller width w4 and the length 12 of the region 506 expands to a greater length 14. In contrast, the NPR region 508 compresses along both the axis of compression and along the perpendicular axis, such that, e.g., both the width w3 and length 13 of the uncompressed NPR region 508 are greater than the width w5 and length 15 of the compressed NPR region 508.

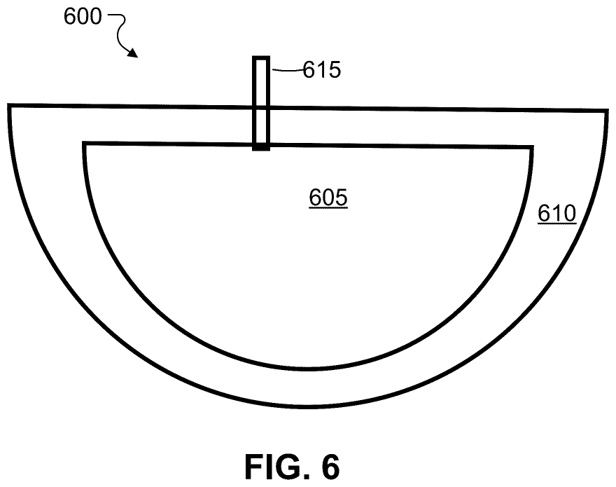

Other electronic devices can benefit from incorporation of NPR materials into their housing. For instance, referring to FIG. 6, a pacemaker 600 includes a hermetic housing 610 with a power pack and one or more processors or controllers 605 disposed in the interior of the hermetic housing 610. In some examples, the power pack 605 is a lithium iodine or lithium-silver vanadium oxide battery. In some examples, the power pack 605 is a radioactive thermoelectric generator. In some examples, the power pack 605 is rechargeable. In some examples, the power pack 605 includes NPR material, e.g., as described for the battery 100 of FIG. 1. An electrode 615 is electrically connected to the power pack 605 and extends through the housing 610 to an exterior of the pacemaker 600 for connection to tissue of a patient.

The pacemaker 600 provides electrical impulses to specified targets. In some examples, the pacemaker 600 is used in the heart, where it delivers electrical pulses to one or more chambers of the heart. In some examples, the pacemaker 600 is used in the brain, where it sends electrical impulses to specified targets in the brain, e.g., to regulate movement control.

The hermetic housing 610 seals the electronic components and circuitry, e.g., the power pack and processors/controllers, into the interior of the pacemaker to prevent them from contacting fluid, e.g., from the patient's tissue. The hermetic housing 610 includes an NPR material. The incorporation of NPR material into the hermetic housing 610 provides the housing with shock absorbing capabilities while reducing the weight of the device as compared to a similar pacemaker having conventional PPR housing. For instance, the hermetic housing 610 can include NPR titanium, aluminum, zirconia, glass, epoxy resins, or polymer materials.

In some examples, the hermetic housing 610 is an NPR-PPR composite, such as a matrix composite, laminar composite, fiber composite, or other appropriately structured composite. includes NPR and PPR materials. The use of NPR-PPR composite materials for the housing 610 provides the frame with advantages stemming from the use of both NPR and PPR materials. For instance, while NPR materials are lightweight, they can be porous and lack hardness. The inclusion of both NPR and PPR materials in the frame allows the density advantages of NPR materials to be achieved while also presenting a non-porous (e.g., water resistant), hard structure via the PPR material of the composite. For instance, the NPR-PPR composite can be structured such that the outward facing surface of the pacemaker is composed of only PPR material, such that the NPR material is not exposed to the external environment. In this arrangement, the weight advantages of NPR material can be achieved while providing robust water resistance and hardness via the exterior PPR surface.

The electrode 615 delivers electrical impulses to tissue of the patient from the pacemaker. In some examples, the electrode 615 includes NPR materials. For instance, the electrode 615 can have a tip formed of NPR material, or can have an NPR material core and a PPR material cladding, as discussed further below. In some examples, the electrode 615 is unipolar, including a single electrical contact at the end of the electrode. In some examples, the electrode 615 is bipolar, including two electrical contacts, one located at the tip and a second at a distance along the length electrode. Having two electrical contacts reduces sensitivity to external interferences. In some examples, the electrode 615 includes materials such as titanium, silicon, polyurethane, or platinum.

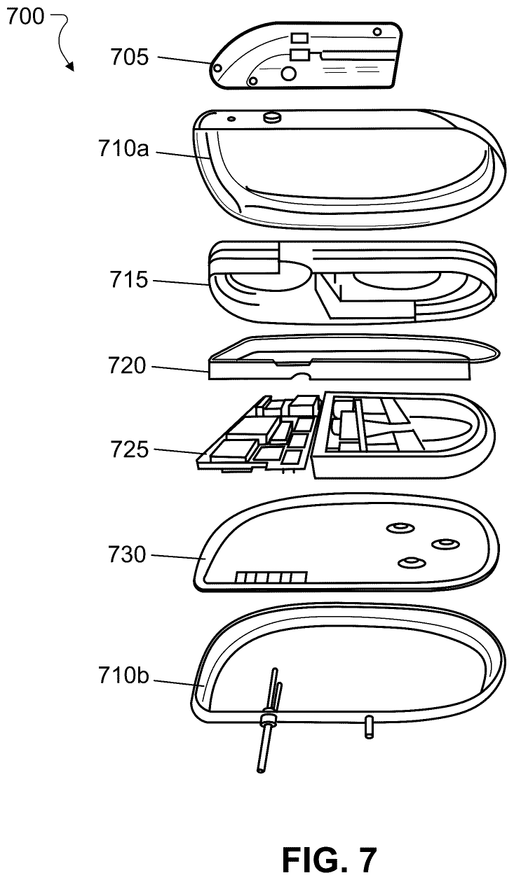

FIG. 7 is an exploded view of a cardiac pacemaker 700. The pacemaker is a layered structure including top and bottom housings 710a, 710b, such as titanium housings. A self-sealing connector 705 is connected to the exterior-facing surface of the top housing 710a and a polypropylene cup 715 is disposed on the interior-facing surface of the top housing 710a. Interior components, including a battery 725, e.g., a lithium-iodine battery, and a radiopaque marker 730, are disposed in the interior space defined by the top and bottom housings 710a, 710b. The top and bottom housings 710a, 710b are secured together with a closure element 720, such as a titanium ring.

The self-sealing connector 705 is an insulated, electrically conductive element that connects the battery 725 to the patient's heart. The self-sealing connector 705 creates a hermetic seal that prevents the entry of bodily fluids or other substances that could compromise the internal components of the cardiac pacemaker 700. In some examples, the self-sealing connector 705 includes NPR material. In some examples, the self-sealing connector 705 includes an NPR-PPR composite material to obtain advantages stemming from both NPR and PPR materials.

The polypropylene cup 715 provides insulation and structural support for the cardiac pacemaker 700. In some examples, the polypropylene cup 715 includes NPR material. As compared to a similar cup composed of only PPR materials, a polypropylene cup 715 with NPR material has lower density, better strength/weight ratio, greater porosity, larger surface area, and better dimensional stability.

The closure element 720 can be a titanium ring that is welded to the housings 710a, 710b to provide a hermetic seal, thus preventing moisture or other substances from entering the cardiac pacemaker 700. In some examples, the closure element 720 and/or the housings 710a, 710b includes NPR material, e.g., NPR-PPR composite materials, to obtain advantages stemming from both NPR and PPR materials.

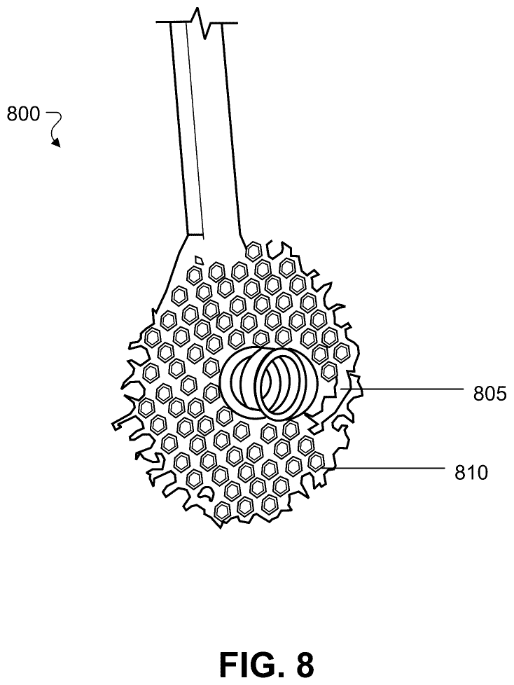

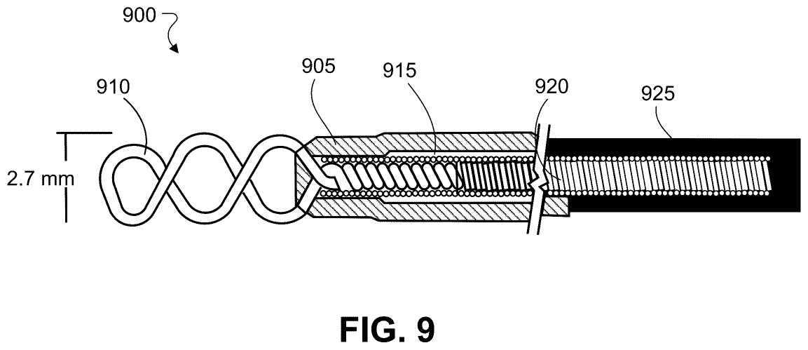

FIGS. 8-9 illustrate example configurations for an electrode tip, such as the tip of a pacemaker electrode (e.g., the electrode 615 of FIG. 6). Referring to FIG. 8, an example pacemaker electrode 800 includes a helically structured coil tip 805 with a mesh substrate 810. The helical shape of the coil tip 805 can improve the precision of electrical stimulation. Additionally, the helical shape provides greater surface area compared to a flat or pointed tip, which can be helpful in applications that may benefit from greater surface area contact, such as when connecting an electrode lead to a cardiac chamber. The mesh substrate 810 is a conductive material that is configured to provide electrical stimulation to specified targets in the heart or brain. The mesh substrate 810 provides electrode surface area that provides areas of contact with surrounding tissue in cardiac chambers or the brain. The mesh substrate 810 also can enhance the flexibility of the pacemaker electrode 800.

Referring to FIG. 9, an example pacemaker electrode 900 includes a coiled inner conductor 915 encapsulated by a hermetic housing 905. A coiled electrode tip 910 is connected to the coiled inner conductor 915 and extends outside the hermetic housing 905. The inner conductor 915 and electrode tip 910 can be, e.g., platinum-iridium, and the housing 905 is a flexible, biocompatible material such as silicone rubber. An electrode wire 918 with a conductive core 920 and an a cladding 925 extends distally away from the inner conductor 915. The electrode wire 918 can be, e.g., eligiloy, and the cladding can be, e.g., stainless steel.

In some electrodes, such as the example electrodes 800, 900 of FIGS. 8-9, the electrode tip (e.g., the tip 802, 910) can include an NPR material, e.g., can be made in part or entirely from NPR material. In some examples, the electrode tip can include a core of NPR material with a PPR coating, such as a coating of platinum black, glassy carbon, or another suitable coating.

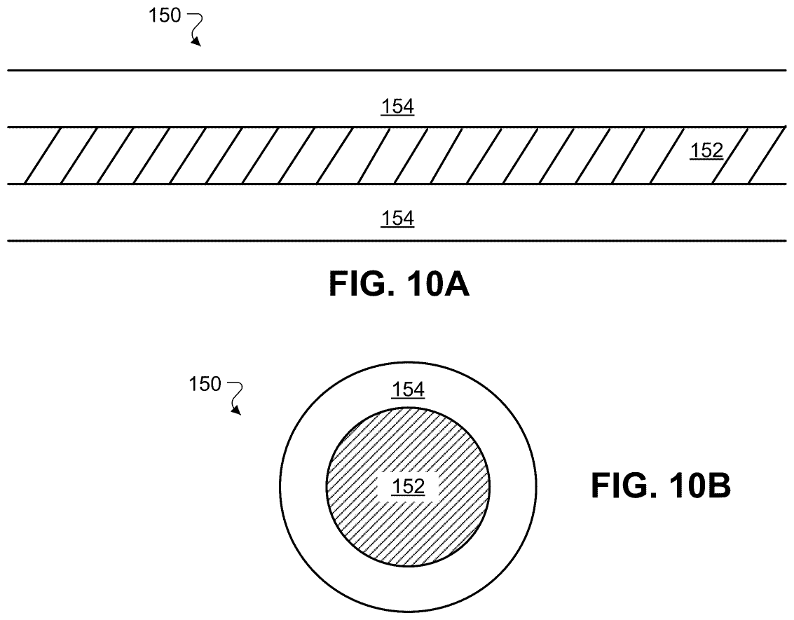

Referring to FIGS. 10A and 10B, in some electrodes, the electrode wire can include an NPR material. FIGS. 10A and 10B show a side cross sectional view and a cross sectional view along the length of an electrode 150. A core 152 of the electrode is composed of an NPR material. The core 152 can be, for instance, an NPR piezoelectric ceramic material; an NPR gold, platinum, or titanium wire; a carbon fiber or graphite core exhibiting a negative Poisson's ratio, or another suitable NPR material. An outer cladding 154 of the electrode is a PPR material, e.g., to provide hardness and biocompatibility. An NPR core of an electrode can be advantageous. For instance, NPR wires have larger surface area and more flexibility than PPR wires and thus can dissipate thermal energy more efficiently. Additionally, a wire with NPR material is less dense and lighter than a wire made from conventional PPR materials.

What is claimed is:

1. A battery comprising:
a frame defining an interior space, the frame comprising:
an inner layer comprising a material having a negative Poisson's ratio (NPR material), and
an outer layer disposed on the inner layer such that the inner layer faces the interior space and the outer layer faces an exterior of the frame;
an electrolyte contained in the interior space of the frame;
a positive electrode disposed in the interior space of the frame, the positive electrode comprising a positive electrode active material; and
a negative electrode disposed in the interior space of the frame, the negative electrode comprising a negative electrode active material.

2. The battery of claim 1, wherein the outer layer of the frame comprises a material having a positive Poisson's ratio (PPR material).

3. The battery of claim 1, wherein the outer layer of the frame comprises an NPR material.

4. The battery of claim 1, wherein the inner layer of the frame comprises a composite material comprising the NPR material and a PPR material.

5. The battery of claim 4, wherein the composite material of the inner layer comprises a matrix of the PPR material with the NPR material embedded therein.

6. The battery of claim 4, wherein the composite material of the inner layer comprises a matrix of the NPR material with the PPR material embedded therein.

7. The battery of claim 4, wherein the composite material of the inner layer comprises a layered composite comprising alternating layers of the PPR material and the NPR material.

8. The battery of claim 1, wherein the inner layer of the frame has a negative Poisson's ratio.

9. The battery of claim 1, wherein the positive electrode active material has a negative Poisson's ratio.

10. The battery of claim 9, wherein the negative electrode active material has a negative Poisson ratio.

11. The battery of claim 1, wherein the positive electrode active material comprises a lithium metal oxide or a lithium metal phosphate.

12. The battery of claim 1, wherein the negative electrode active material comprises graphite, graphene, or a nanostructured carbon material.

13. The battery of claim 1, comprising a porous membrane disposed in the interior space between the positive electrode and the negative electrode.

14. The battery of claim 13, wherein the porous membrane comprises an NPR material.

15. A pacemaker comprising:
a hermetic housing comprising a material having a negative Poisson's ratio (NPR material);
a power pack disposed in an interior of the hermetic housing, wherein the power pack is configured to generate electrical pulses; and
an electrode, wherein a first end of the electrode is electrically connected to the power pack and a second end of the electrode extends through the hermetic housing to an exterior of the pacemaker, the second end of the electrode configured to be connected to tissue of a patient.

16. The pacemaker of claim 15, wherein the hermetic housing comprises a composite material comprising the NPR material and a PPR material.

17. The pacemaker of claim 16, wherein the composite material of the hermetic housing comprises a matrix of the PPR material with the NPR material embedded therein.

18. The pacemaker of claim 16, wherein the composite material of the hermetic housing comprises a matrix of the NPR material with the PPR material embedded therein.

19. The pacemaker of claim 16, wherein the composite material of the hermetic housing comprises a layered composite comprising alternating layers of the PPR material and the NPR material.

20. The pacemaker of claim 16, wherein an outer surface of the hermetic housing is formed of PPR material.

21. The pacemaker of claim 15, wherein the electrode comprises an NPR material.

22. The pacemaker of claim 21, wherein the electrode comprises:
a core comprising the NPR material; and
a coating surrounding a length of the core, the coating comprising a PPR material.

23. The pacemaker of claim 15, wherein a distal tip of the electrode comprises an NPR material.
